(19) United States
(12) Patent Application Publication
(10) Pub. No.: US 2026/0020805 A1
(43) Pub. Date: Jan. 22, 2026

(54) WEARABLE DEVICES FOR MONITORING BRAIN ACTIVITY AND PROVIDING NON-INVASIVE STIMULATION AND METHODS OF IMPLEMENTATION THEREOF

(71) Applicant: BRAINPATCH LTD, London (GB)

(72) Inventors: Nickolai Vysokov, London (GB); lllya Tarasenko, London (GB); Dauren Toleukhanov, London (GB); Mikheil Oganesyan, London (GB)

(21) Appl. No.: 19/346,705

(22) Filed: Oct. 1, 2025

Related U.S. Application Data
(63) Continuation-in-part of application No. 17/413,310, filed on Jun. 11, 2021.

(51) Int. Cl. A61B 5/291; A61B 5/375; A61B 5/377; A61N 1/04; A61N 1/36

(52) U.S. Cl. CPC A61B 5/291; A61B 5/375; A61B 5/377; A61N 1/0484; A61N 1/36031

(57) ABSTRACT

Disclosed is wearable device for monitoring brain activity and for providing non-invasive stimulation, wearable device comprising: headwear arrangement comprising: electrode arrangement comprising electrodes configured to make electrical contact with skin of user; and flexible cushioning member supporting electrodes; input/output (I/O) arrangement configured to: receive electrical signals (ES) from electrode(s); transmit ES to data processing arrangement (DPA) to process ES for generating brain stimuli (BS); and generate and apply BS generated using a brain stimulation protocol (BSP) to electrode(s), BS applied on skin lying partially in mastoid region; DPA comprising processing unit configured to: receive ES from I/O arrangement; analyse ES with predetermined reference data set, by extracting signal feature(s); apply processing algorithm(s), to map signal feature(s) to stimulation parameter(s); generate BSP comprising stimulation parameter(s); and transmit BSP to I/O arrangement; and power unit(s) supplying electrical power to I/O arrangement and DPA.

### CROSS-REFERENCE TO RELATED APPLICATIONS

This application is a continuation-in-part of U.S. patent application Ser. No. 17/413,310, titled “BRAIN INTERFACING APPARATUS AND METHOD” filed on 13 Dec. 2019 and U.S. patent application Ser. No. 17/563,621, titled “WEARABLE ELECTRODE ARRANGEMENT” filed on 28 Dec. 2021, which is incorporated herein by reference.

### TECHNICAL FIELD

The present disclosure relates to wearable devices for monitoring brain activity and for providing non-invasive stimulation. Moreover, the present disclosure relates to methods of monitoring brain activity and providing non-invasive stimulation, the method being implemented by wearable devices.

### BACKGROUND

Presently, wearable devices used for health monitoring and neuromodulation are increasingly being explored to support human wellness by monitoring physiological activity and by providing external stimulation to influence bodily functions. Such devices belong to the field of biomedical engineering and human-computer interaction, where devices are designed to non-invasively interact with a user's body in order to collect physiological data or to provide therapeutic interventions.

Despite advancements, several persistent problems remain unaddressed in a domain of brain monitoring and stimulation. Typically, wearable devices often suffer from poor signal quality, particularly due to unstable or inconsistent contact between electrodes and head of a user. This results in inaccurate readings, loss of critical data, and reduced effectiveness of brain activity analysis. Moreover, existing devices apply generic electrical signals, thus not able to regulate a nervous system of individual users, which leads to suboptimal therapeutic outcomes, reduced user comfort, and increased risks of non-compliance during prolonged usage.

However, existing wearable devices are associated with several limitations. Firstly, the wearable devices rely on rigid placement of the electrodes that provide fixed contact points, which are often uncomfortable for the users and do not adapt to natural movement of the user, resulting in signal artefacts. Secondly, the wearable devices employ dry-contact electrodes, which eliminate the need for gels but suffer from high impedance and unreliable signal acquisition. Thirdly, the wearable device relies on general-purpose neurostimulation protocols, which are not dynamically adjusted to reflect a changing physiological state of the user, thereby limiting therapeutic benefit.

### SUMMARY

The aim of the present disclosure is to provide a wearable device for monitoring brain activity and for providing non-invasive stimulation and a method for monitoring a brain activity and providing non-invasive stimulation, the method being implemented by a wearable device to ensure reliable acquisition of electrical signals corresponding to a brain activity of an user and controlled application of brain stimuli based on brain stimulation protocol. The aim of the present disclosure is achieved by a wearable device for monitoring brain activity and for providing non-invasive stimulation and a method for monitoring a brain activity and providing non-invasive stimulation, the method being implemented by a wearable device as defined in the appended independent claims to which reference is made to. Advantageous features are set out in the appended dependent claims.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

Embodiments of the present disclosure will now be described, by way of example only, with reference to the following diagrams wherein:

FIG. 1A illustrates a schematic illustration of a wearable device for monitoring brain activity and for providing non-invasive stimulation, and

FIG. 1B illustrates an exemplary implementation of a headwear arrangement in use, in accordance with an embodiment of the present disclosure;

FIGS. 2A, 2B, 2C, and 2D collectively illustrate different perspective views of placement of a portion of a wearable device over head of a user in accordance with an embodiment of the present disclosure;

FIG. 3 illustrates an exemplary implementation of a wearable device for monitoring brain activity and for providing non-invasive stimulation, in accordance with an embodiment of the present disclosure; and

FIGS. 4A and 4B collectively illustrate steps of a method for monitoring a brain activity and providing non-invasive stimulation, the method being implemented by a wearable device, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In a first aspect, the present disclosure provides a wearable device for monitoring brain activity and for providing non-invasive stimulation, the wearable device comprising:
a headwear arrangement comprising an electrode arrangement having a plurality of electrodes, wherein when the headwear arrangement is worn over a head of a user, the plurality of electrodes are configured to make electrical contact with a skin of a user, the skin lying at least partially in a mastoid region of the user;
an input/output arrangement that is operably coupled with the plurality of electrodes, wherein, when in operation, the input/output arrangement is configured to:
   receive electrical signals from at least one of the plurality of electrodes, wherein the electrical signals correspond to at least one of: the brain activity of the user, vagus nerve activity of the user;
   transmit the electrical signals to a data processing arrangement to process the electrical signals for generating a brain stimuli, based on a brain stimulation protocol; and
   generate and apply the brain stimuli to the at least one of the plurality of electrodes, the brain stimuli being generated using the brain stimulation protocol received from the data processing arrangement, wherein the brain stimuli are applied on the skin lying at least partially in the mastoid region;
the data processing arrangement communicably coupled with the input/output arrangement, the data processing arrangement comprising a processing unit, wherein the processing unit is configured to:
   receive the electrical signals from the input/output arrangement;
   analyse the electrical signals;
   apply at least one processing algorithm, to map one or more signal features to one or more stimulation parameters;
   generate the brain stimulation protocol comprising the one or more stimulation parameters; and
   transmit the brain stimulation protocol to the input/output arrangement.

The aforementioned first aspect describes the wearable device for monitoring the brain activity and for providing the non-invasive stimulation, thereby enabling a closed-loop system. Herein, the headwear arrangement comprising the electrode arrangement and the flexible cushioning member, maintains stable electrical contact between the plurality of electrodes and the skin of the user at the mastoid region, thereby improving signal quality and ensuring reliable acquisition of the electrical signals corresponding to the brain activity of the user. The input/output arrangement operably coupled with the plurality of electrodes ensures that the electrical signals are accurately received, conditioned, and transmitted to the data processing arrangement, thereby enabling the processing unit to analyse the electrical signals against the predetermined reference data set. Moreover, through an extraction of one or more signal features and an application of the at least one processing algorithm, the processing unit maps the signal features to one or more stimulation parameters for generating the brain stimulation protocol. The brain stimulation protocol is then communicated back to the input/output arrangement to generate and apply the brain stimuli at the mastoid region of the user.

Moreover, the wearable device enables modulation of autonomic balance between a sympathetic division and a parasympathetic division of a nervous system, without requiring invasive procedures. Additionally, the inclusion of the one or more power units provides a reliable and portable source of electrical power to the input/output arrangement and the data processing arrangement, thereby ensuring uninterrupted operation of the wearable device.

In a second aspect, the present disclosure provides a method for monitoring a brain activity and providing non-invasive stimulation, the method being implemented by a wearable device, the method comprising:
positioning a headwear arrangement over a head of a user, the headwear arrangement comprising a plurality of electrodes configured to make electrical contact with skin of the user lying at least partially in a mastoid region of the user;
receiving, by the input/output arrangement that is operably coupled with the plurality of electrodes, electrical signals from at least one of the plurality of electrodes, wherein the electrical signals correspond to at least one of: brain activity of the user, heart activity of the user, vagus nerve activity of the user;
transmitting, by the input/output arrangement, the electrical signals to the data processing arrangement;
analysing, by the data processing arrangement, the electrical signals;
applying, by the data processing arrangement, at least one processing algorithm to map the one or more signal features to one or more stimulation parameters in a brain stimulation protocol;
generating, by the data processing arrangement, a brain stimulation protocol comprising the one or more stimulation parameters;
transmitting, by the data processing arrangement, the brain stimulation protocol to the input/output arrangement; and
generating and applying, by the input/output arrangement, the brain stimuli to the at least one of the plurality of electrodes using the brain stimulation protocol, the brain stimuli being applied on the skin lying at least partially in the mastoid region.

The aforementioned second aspect describes a method for monitoring the brain activity and providing non-invasive stimulation, implemented by the wearable device. Herein, there is reliable acquisition of electrical signals corresponding to the brain activity of the user through the positioning of the headwear arrangement over the head of the user, with plurality of electrodes configured to make electrical contact with skin of the user lying at least partially in the mastoid region of the user. Moreover, receiving and transmitting the electrical signals from the input/output arrangement to the data processing arrangement, is useful for analysing the electrical signals with respect to the predetermined reference data set, to determine a variation of the electrical signals. Herein, through the extraction of one or more signal features and application of at least one processing algorithm, the one or more signal features are mapped to one or more stimulation parameters, thus allowing dynamic adaptation of the brain stimulation protocol. This dynamic adaptation provides a balance between the sympathetic division and the parasympathetic division of a nervous system. The brain stimuli, generated using the brain stimulation protocol, are then applied to the skin in the mastoid region. This non-invasive stimulation enables modulation of autonomic balance, contributing to therapeutic outcomes. Moreover, the one or more power units ensures a portable, reliable power source for supporting uninterrupted operation of the wearable device.

Throughout the present disclosure, the term “monitoring brain activity” refers to monitoring of electrical signals received from a brain of the user by a method of electroencephalography (EEG). Additionally, the monitoring may include electrical signals corresponding to vagus nerve activity, wherein the electrodes positioned at least partially in the mastoid region are configured to detect electrical activity from the auricular branch of the vagus nerve. The electrical signals may thus reflect both brain activity and peripheral nervous system activity relevant to autonomic regulation. Optionally, the monitoring of brain activity may include detection of electrical signals which include, but are not limited to, signals, or a combination of signals, obtained using electric field encephalography (EFEG), near infrared spectroscopy (NIRS), Magnetoencephalography (MEG), electromyography (EMG), electrocardiogramaye tracking and/or functional magnetic resonance imaging (fMRI). Moreover, optionally, the monitoring of the brain activity relates to monitoring of a change in electrical activity of the brain of the user.

Throughout the present disclosure, the term “headwear arrangement” refers to a portion of the wearable device that is configured to be worn by the user on his/her head. Examples of the headwear arrangement may include, but are not limited to, a cap, a hat, a helmet, headphones, a headband, a pair of glasses, and a bonnet. The headwear arrangement could be fabricated using an electrically insulating material. Examples of the electrically insulating materials may include, but are not limited to, wool, cotton, polyester, rubber, lycra, nylon, and buckram. Optionally, the headwear arrangement is ergonomically shaped to position the plurality of electrodes over the mastoid region of the user without requiring precise alignment, thereby maintaining reliable and comfortable contact between the plurality of electrodes and the skin of the user. Moreover, the electrode arrangement is arranged in such a manner that the skin lying at least partially in the mastoid region of the user provides a stable and comfortable placement point that avoids interference from hair-covered scalp regions.

Throughout the present disclosure, the term “mastoid region” refers to a bony area located posterior to an auricle of an ear, which provides a relatively flat, hair-free surface for the electrode arrangement. The mastoid region is selected for the electrode arrangement because it provides a consistent access to a cranial site of the mastoid region of the user suitable for the non-invasive stimulation and for the monitoring of the brain activity. Moreover, the mastoid region of the user offers comfort during prolonged wear of the headwear arrangement, thereby reducing risk of non-compliance.

Throughout the present disclosure, the term “electrode” refers to an electrical conductor, wherein the electrode is operable to convert an ionic potential at skin of the user into an electric potential and to induce electromagnetic fields on the head of the user, within a skull of the user. Moreover, the term “electrode arrangement” refers to a configuration of the plurality of electrodes that is supported by the headwear arrangement and positioned to make electrical contact with the skin of the user lying at least partially in the mastoid region. The electrode arrangement is configured to enable both the monitoring of the brain activity and for providing the non-invasive stimulation to the mastoid region of the user.

The electrode arrangement is a non-invasive type (for example, such as surface electrodes). The plurality of electrodes of the electrode arrangement are operatively coupled with one or more components of the wearable device. Each of the plurality of electrodes are manufactured using an electrode material. Examples of with the electrode material may include, but are not limited to, stainless steel, platinum, sliver chloride-coated silver, carbon rubber, and graphene.

Herein, the plurality of electrodes of the electrode arrangement is placed or positioned by the headwear arrangement such that the plurality of electrodes make an electrical contact with a skin of a user, the skin lying at least partially in the mastoid region of the user. In an instance, the plurality of electrodes may optionally be configured as an EEG electrode arrangement. Such arrangements are well-known in the art. The plurality of electrodes are hybrid electrodes which can function as both for EEG recording and/or for non-invasive stimulation that is provided electrically, wherein the non-invasive stimulation comprises at least one of: transcranial current stimulation (tCS), transcranial direct current stimulation (tDCS), transcranial alternating current stimulation (tACS), transcranial random noise stimulation (tRNS), transcranial temporal interference stimulation (TI), transcranial temporal summation (TS), any other arbitrary transcranial electric current stimulation (tES) protocol generated by the at least one processing algorithm.

Throughout the present disclosure, the term “flexible cushioning member” refers to a compliant structure disposed between the headwear arrangement and the plurality of electrodes, the flexible cushioning member being configured to maintain physical contact between the plurality of electrodes and the skin of the user. Herein, by biasing the plurality of electrodes into gentle contact with the skin of the user, the flexible cushioning member provides a stable, low-impedance interface necessary for monitoring brain activity and for reproducible operation of the wearable device.

Throughout the present disclosure, the term “input/output arrangement” refers to a programmable and/or a non-programmable component of the wearable device that is operably coupled with the plurality of electrodes, and is configured, when in operation, to receive, modify, convert, process or generate electrical signals from at least one of the plurality of electrodes. Optionally, the input/output arrangement is implemented as a hardware or a software, or a combination thereof. In an implementation, the input/output arrangement comprises low-noise amplifiers, filters, and an analog to digital converter, to detect the electrical signals from the at least one of the plurality of electrodes which are weak and filter them to suppress noise. Moreover, optionally, the input/output arrangement further comprises at least one multiplexer to select channel and a circuit for monitoring impedance of the plurality of electrodes. The input/output arrangement ensures that the electrical signals which are stable and reliable corresponding to the brain activity of the user are acquired.

Throughout the present disclosure, the term “data processing arrangement” refers to programmable and/or non-programmable components that are configured to receive the electrical signals corresponding to the brain activity of the user from the input/output arrangement and process the electrical signal for generating the brain stimuli. Optionally, the data processing arrangement can include, for example, a component included within an electronic communications network. Moreover, the data processing arrangement may include hardware, software, firmware or a combination of these, suitable for storing and processing various information and services accessed by one or more user using one or more user equipment. Moreover, the data processing arrangement may include functional components, for example, a processor, a memory, a network adapter and so forth. For example, the data processing arrangement can be implemented using a computer, a phone (for example, a smartphone), a local server, a server arrangement (such as, an arrangement of two or more servers communicably coupled with each other), a cloud server, a quantum computer and so forth.

The data processing arrangement processes the electrical signals received from the input/output arrangement by extracting one or more features from the electrical signals, including, for example, spectral band powers, event-related potentials, or physiological markers derived from the electrical signals such as heart rate variability indices. The one or more features of the electrical signals is compared against stored information or datasets maintained by the data processing arrangement, which can be updated during operation, to determine deviations from a desired state. Moreover, based on the comparison, the data processing arrangement computes one or more stimulation parameters including, but not limited to, an amplitude, a frequency, a time-period, a phase, or a duty cycle.

Throughout the present disclosure, the term “brain stimuli” refers to a current signal that is provided as any one of: a single defined sequence, multiple defined sequences, of the current signal amplitudes delivered via the input/output arrangement to at least one of the plurality of electrodes applied to the skin of the user, in order to modify and/or enhance an activity of the current signal in the brain of the user or in nervous tissues of the user. When in use, when the brain stimuli is applied to the skin lying at least partially in the mastoid region, electric fields are generated from the point of contact of the at least one of the plurality of electrodes, on the skin. The terms ‘brain stimulus’ (singular) and ‘brain stimuli’ (plural) are used interchangeably throughout this disclosure to refer to the electrical stimulation applied to the user, with the singular form used when referring to a single application or instance of stimulation, and the plural form used when referring to multiple applications or the general concept of the stimulation.

Throughout the present disclosure, the term “brain stimulation protocol” refers to a set of information generated by the data processing arrangement defining one or more stimulation parameters for generating the brain stimuli. The brain stimulation protocol comprises electrical signal characteristics for at least one of the plurality of electrodes that is positioned to make contact with the skin lying at least partially in the mastoid region. Optionally, the set of information comprises at least one of: an amplitude of current signal, a time-period of an application of the brain stimuli, a phase of the current signal, one or more frequencies of the brain stimuli and power of the one or more frequencies. The set of information gives rise to a specific sequence of the brain stimuli that is to be generated. The generated brain stimuli will be in the form of any one of: a defined sequence, multiple sequences, of the current signal amplitudes between a combination of the plurality of electrodes (for example, such as at least one pair of electrodes).

The input/output arrangement, upon receiving the brain stimulation protocol from the data processing arrangement, is configured to generate the brain stimuli in accordance with the one or more stimulation parameters defined in the brain stimulation protocol. The brain stimuli are applied to at least one of the plurality of electrodes. Herein, the plurality of electrodes are in the electrical contact with the skin of the user lying at least partially in the mastoid region, and the brain stimuli creates electric fields directed toward neural pathways in the mastoid region without penetrating a cranium to any significant degree, providing non-invasive stimulation distinct from transcranial stimulation. Optionally, the brain stimuli targets at least an auricular branch of the vagus nerve, which is located in proximity to the mastoid region. Herein, the non-invasive stimulation includes the mastoid region where the auricular branch of the vagus nerve exits the cranium by passing through a tympanomastoid fissure between the mastoid region and a tympanic part of temporal bone, and divides into two branches, one joining a posterior auricular nerve and the other extending to the skin of the auricle and a posterior part of an ear canal. Optionally, the brain stimuli also produce electric fields in adjacent neural structures, such as vestibular nerve, which communicates with brain stem and thalamic relay nuclei, thereby providing additional or fallback simulation pathways. Optionally, the brain stimuli targets a vestibular nerve.

Advantageously, applying the brain stimuli to the skin lying at least partially in the mastoid region produces multiple benefits comprises at least one of: an improvement of spontaneous cardiac baroreflex sensitivity, shifting cardiac autonomic function toward parasympathetic predominance, reducing sympathetic overactivity in conditions such as cardiac failure.

Optionally, an activation of the vagus nerve by applying the brain stimuli on the skin lying partially in the mastoid region engages a cholinergic anti-inflammatory pathway and a hypothalamic pituitary adrenal axis, thereby modulating immune responses and attenuating inflammation. Optionally, by targeting both the vagus and the vestibular nerves, the brain stimuli also exert beneficial effects on the brain processes related to cognitive functioning, while simultaneously reducing stress impacts and alleviating emotional burnout. Moreover, applying the brain stimuli to the skin lying at least partially in the mastoid region has been shown to enhance creative thinking, internally oriented attention, and brain resource availability. Beneficially, the application of the brain stimuli through the mastoid region provides a safe, effective, and non-invasive approach for influencing autonomic balance, brain activity, and related physiological functions of the user.

The data processing arrangement is communicably coupled with the input/output arrangement. Herein, the data processing arrangement comprises the processing unit configured to receive the electrical signals corresponding to the brain activity of the user from the input/output arrangement. The data processing arrangement is communicably coupled with the input/output arrangement through a wired communication link (for example, such as a conductive connector, a serial bus, and the like) or a wireless communication link (for example, such as wireless protocols such as Bluetooth®, Wi-Fi). Moreover, the processing unit comprises one or more programmable or one or more non-programmable components, such as a microprocessor, a digital signal processor, or a dedicated integrated circuit, that are operable to accept the electrical signals after conditioning and digitizing by the input/output arrangement. Herein, the processing unit ensures that accurate and noise-related data reflecting real-time brain activity of the user is made available for further analysis.

Throughout the present disclosure, the term “predetermined reference data set” refers to collection of reference data derived from the monitoring of the brain activity from a plurality of persons. Herein, the plurality of persons may be of various age groups, sex, mental health condition, and physical health condition, and geographical location. Optionally, the predetermined reference data set also comprises baseline recording of the monitored brain activity of the user, wherein such a baseline recording is captured during at least one of: a resting state, a neutral state, a working state, such that the user's brain activity is compared against their own baseline recording.

Throughout the present disclosure, the term ‘signal features’ or ‘one or more signal features’ refers to characteristics extracted from the electrical signals that are used for analysis and mapping to stimulation parameters. The signal features may include, but are not limited to, frequency components, amplitude variations, spectral band powers, event-related potentials, phase relationships, or time-domain characteristics of the electrical signals.

The processing unit of the data arrangement analyses the electrical signal received from the input/output arrangement using the predetermined reference data set, by extracting one or more signal features. The analysis is performed by comparing the one or more signals features of the electrical signals against corresponding features in the predetermined reference data set, thereby allowing the processing unit to identify deviations from a physiological state or a cognitive state of the baseline recording of the user. Moreover, the analysis of the electrical signals provides information required for computing one or more stimulation parameters.

The processing unit applies at least one processing algorithm to map the one or more signal features to the one or more stimulation parameters. Moreover, the processing unit ensures that the generated brain stimuli are suitable for providing the balance between the sympathetic division and the parasympathetic division of the nervous system by mapping the one or more signal features to the one or more stimulation parameters. Optionally, the at least one processing algorithm is implemented as at least one of: executable software, digital hardware. Examples of digital hardware may include, but are not limited to, FPGA, ASIC, custom chip design.

In an exemplary implementation, the brain stimulation protocol generated by the processing unit is configured as Vagus Nerve Stimulation (VNS) protocol that influences an emotional state of the user by modulating the activation-related neurodynamic. Moreover, the mapping of the one or more signal features to the one or more stimulation parameters is configured to determine the effects of the brain stimuli on the balance between the sympathetic division and the parasympathetic division of the nervous system, as well as on the psychological well-being of the user.

The processing unit of the data processing arrangement, after applying the at least one processing algorithm to the electrical signals received from the input/output arrangement, generates the brain stimulation protocol. The brain stimulation protocol comprises the one or more stimulation parameters. Herein, once generated, the brain stimulation protocol is transmitted by the processing unit to the input/output arrangement. Moreover, the brain stimulation protocol is transmitted via a wired communication link or wireless communication link internal to the headwear arrangement, ensuring that the input/output arrangement receives the one or more stimulation parameters generated by the data processing arrangement. Furthermore, the input/output arrangement uses the one or more stimulation parameters to generate and apply the brain stimuli to the at least one of the plurality of electrodes positioned on the mastoid region of the user. Herein, the generation of the brain stimulation protocol is essential to translate the electrical signal corresponding to the brain activity of the user into the one or more stimulation parameters, ensuring that the applied brain stimuli is precise, adaptive and safe.

In an exemplary implementation, the plurality of electrodes may be positioned on the skin of the user lying at least partially in the mastoid region of the user. Herein, at least one of the plurality of electrodes may be configured to receive electrical signals corresponding to the brain activity of the user. As an example, the electrical signals may include electroencephalography (EEG) signals. The electrical signals may be transmitted to the processing unit of the data processing arrangement, wherein the processing unit may extract one or more signal features from the electrical signals. The one or more signal features may comprise frequency component in an alpha band, wherein the frequency component lies in a range of 8 hertz (Hz) to 12 Hz. Herein, a predetermined reference data set may be used for comparison, wherein, the processing unit may compare the one or more signal features against a reference frequency component comprised in the predetermined reference data set to identify a deviation from a desired physiological state. Thereafter, the processing unit may apply an algorithm (for example, such as a neural network model) to dynamically determine the one or more stimulation parameters suitable to restore balance between the sympathetic division and the parasympathetic division of the nervous system. Accordingly, the processing unit may generate the brain stimulation protocol that defines the one or more stimulation parameters for implementation by the input/output arrangement. Herein the brain stimulation protocol may comprise use of a left electrode of the plurality of electrodes corresponding to the mastoid region of the user, a biphasic rectangular waveform, a stimulation frequency, a pulse width, and a duty cycle. Subsequently, the brain stimulation protocol is transmitted by the processing unit of the data processing arrangement to the input/output arrangement. Consequently, the input/output arrangement generates and applies the brain stimuli to in the mastoid region of the user via the left electrode.

Throughout the present disclosure, the term “power unit” refers to a power source being configured to provide electrical power to the input/output arrangement and the data processing arrangement. Optionally, the one or more power units may include one or more cells or one or more batteries capable of providing electrical power. In an example, the power unit may provide 12 volts (V) electrical supply to the input/output arrangement and 5 V electrical supply to the data processing arrangement. The one or more power units are configured supply electrical power to the input/output arrangement and the data processing arrangement, when in operation. Optionally, the one or more power units may include at least one of the following sources including, but not limited to: a solid-state battery (for example, a ceramic-based battery, a glass-based battery or a sulphide-based battery) and a lithium-ion battery (Li-ion) or lithium-polymer battery (Lipo), as well as a generator of power from sources like movement or solar energy, a receiver for one of wireless power transfer technologies, or a surge protecter. Optionally, the wearable device comprises of at least two power units for providing an isolated electrical power to the input/output arrangement responsible for recording or monitoring and processing of electrical signal received form the brain of the user) and an output portion (comprising of units/arrangements responsible for the generation of the brain stimuli), respectively.

In an exemplary implementation, the one or more power units comprises a battery source of 3.7 V Li-ion cell, that serves as a supply of power. The one or more power units further comprises a power boost generator and a regulator circuit, which are configured to the 3.7 V input from the Li-ion cell into a 5 V supply for powering the operation of the wearable device and a 15 V supply for the input/output arrangement. Moreover, the one or more power units may comprise a voltage splitter to provide a differential supply, wherein the differential supply lies in a range of +/−12-40 V.

Optionally, the data processing arrangement further comprises a memory module that stores the predetermined reference data set, wherein the processing unit is further configured to iteratively update the predetermined reference data set in the memory module, when applying the at least processing algorithm. In this regard, the term “memory module” refers to a volatile storage medium or non-volatile storage medium, such as an electrical circuit, a magnetic disk, a virtual memory or an optical disk, in which a computer and/or the data processing arrangement may store data for any duration. Optionally, the memory module may be a non-volatile mass storage such as physical storage media. Moreover, the memory module stores the predetermined reference data set. Herein, an operation of the memory module may include updating the predetermined reference data set based on the electrical signals or the one or more stimulation parameters derived from the electrical signals received from the brain of the user, by storing the received electrical signals or the stimulation parameters in the memory module during the operation. Herein, the processing unit extracts the one or more signal features from the electrical signals and compares the electrical signals with corresponding features in the predetermined reference data set to identify deviations from the desired state. The processing unit further applies the at least one processing algorithm, during which the predetermined reference data set stored in the memory module is iteratively updated with newly derived features, thereby refining or expanding the predetermined reference data set. The predetermined reference data set is updated, ensuring that said predetermined reference data set remains representative of evolving brain activity of the user. A technical effect of the aforementioned feature is that it ensures that the brain stimulation protocol which is generated in real time reflects physiological changes of the user and improves the effectiveness of the applied brain stimuli in restoring balance between division the sympathetic and parasympathetic division of the user's nervous system.

Optionally, when in operation, the input/output arrangement is configured to receive the electrical signals from the at least one of the plurality of electrodes concurrently with generating and applying the brain stimuli to the at least one of the plurality of electrodes. In this regard, the data processing arrangement analyses the received electrical signals and generates the brain stimulation protocol in a concurrent manner, when the electrical signals are received from the input/output arrangement and when the brain stimuli are applied by the input/output arrangement to the at least one of the plurality of electrodes. Herein, the term “concurrent manner” refers to receiving the electrical signals and generating the brain stimulation protocol in a particular time period, such that both actions appear simultaneous in nature. This concurrent operation is achieved by equipping the input/output arrangement with parallel functional blocks, for example, such as, low-noise amplifiers, filters, and analog to digital converters. Such parallel functional blocks enable acquisition of weak electrical signals while at same time providing waveforms of the brain stimulation protocol through the input/output arrangement. Moreover, the input/output arrangement enables real time or near-real time closed loop monitoring, wherein the electrical signals received from the at least one of the plurality of electrodes are continuously analysed by the data processing arrangement to adjust the brain stimulation protocol during ongoing delivery of the brain stimuli on the skin lying at least partially in the mastoid region.

A technical effect of the aforementioned feature is that it provides continuous and adaptive control of the application of the brain stimuli without interruption of monitoring, thereby reducing latency, improving responsiveness to physiological changes, and ensuring that the stimulation remains optimized to maintain balance between the sympathetic division and the parasympathetic division of the nervous system. This results in enhanced therapeutic precision, safety, and user comfort, as the brain stimulation protocol can be updated dynamically based on the real-time psychological state of the user.

Optionally, when in operation, the processing unit is configured to analyse the electrical signals with the predetermined reference data set concurrently with the application of the brain stimuli to the at least one of the plurality of electrodes, by configuring the input/output arrangement.

In this regard, the concurrent operation is enabled by the configuring the input/output arrangement to both deliver the brain stimuli and provide real-time acquisition of the electrical signals. Herein, when in operation, the processing unit continuously extract one or more signals features from the electrical signals and compares the one or more signal features with the corresponding features in the predetermined reference data set. Moreover, this comparison of the electrical signals with the predetermined reference data set, allows the processing unit to assess whether the applied brain stimuli are achieving the desired modulation of the brain activity and autonomic balance, or whether an update to the brain stimulation protocol is required.

A technical effect of the aforementioned features is that it allows closed loop operation in which analysis of the electrical signals with the predetermined reference data set occurs continuously during stimulation, thereby reducing latency, enabling rapid adaptation to physiological changes, and ensuring that the generated brain stimulation protocol remains optimized to provide a balance between the sympathetic division and the parasympathetic division of the nervous system.

Optionally, the processing unit is further configured to monitor one or more physiological markers corresponding to the brain stimuli, the one or more physiological markers comprising at least one of: a heart rate variability parameter, a beat-to-beat interval, a ratio of low frequency to high frequency. In this regard, the term “heart rate variability parameter” (HRV) refers to a measure of variation in time interval between consecutive heartbeats, reflecting autonomic nervous system dynamics. The HRV is recognized as an indicator of psychological stress, where chronic stress leads to hyperactivation of the sympathetic division of the nervous system. A higher HRV is indicative of greater adaptability and resilience to stress, whereas a reduced HRV is a marker of vulnerability to stress and disease, and may reflect diminished vagal activity. Moreover, the term “beat-to-beat-interval” refers to a time interval between two successive R-waves in ECG or any equivalent measurement of cardiac electrical activity, corresponding to heartbeats. Moreover, the time interval is used by the processing unit to derive HRV parameters and to provide fine-grained monitoring of cardiac responses to the applied brain stimuli. Furthermore, term “ratio of low frequency to high frequency” (LF/HF ratio) refers to a ratio of power spectral density of low-frequency components to that of high-frequency components in HRV analysis. The LF/HF ratio is a standard metric representing the balance between sympathetic division activity and parasympathetic division activity, with elevated LF/HF ratios indicating sympathetic predominance and reduced ratios indicating parasympathetic predominance.

Herein, the processing unit is configured to receive physiological markers via the input/output arrangement concurrently with or after application of the brain stimuli. The processing unit extracts the beat-to-beat interval from these signals, computes the heart rate variability parameter in either time domain or in frequency domain, and calculates the ratio of low frequency to high frequency. Moreover, the physiological markers are then compared against the predetermined reference data set. The deviations from the predetermined reference data sets are used by the processing unit to update the brain stimulation protocol by adjusting one or more stimulation parameters of the brain stimuli. The processing unit monitors the physiological markers because they serve as objective indicators of whether the brain stimuli applied at least partially in the mastoid region of the user is achieving the desired autonomic modulation. For example, an increase in HRV or a shift in the LF/HF ratio toward parasympathetic predominance indicates that the stimulation is effective in reducing sympathetic overactivity and improving stress resilience.

A technical effect of monitoring the one or more physiological markers in such a manner is that provides a closed-loop feedback mechanism wherein the effectiveness of the applied brain stimuli are validated and dynamically adjusted using real-time monitoring of HRV parameters, beat-to-beat intervals, and LF/HF ratios, ensuring that the one or more stimulation parameters is safe, adaptive, and personalised, resulting in improved restoration of autonomic balance between the sympathetic division and the parasympathetic division of the nervous system, reduced psychological stress, enhanced recovery capacity, and increased resilience against stress-related disorders.

Optionally, the at least one processing algorithm comprises at least one of: at least one adaptive learning algorithm, at least one computational algorithm. In this regard, the processing unit generates the brain stimulation protocol by implementing the adaptive learning algorithms or the computational algorithms after analysing the electrical signals received from the input/output arrangement. The term “adaptive learning algorithm” refers to machine learning or artificial intelligence-based algorithm configured to iteratively adjust one or more stimulation parameters of the brain stimulation protocol based on real-time analysis of the electrical signals received from the plurality of electrodes. The at least one adaptive learning algorithm is operable to identify patterns in the one or more signal features, compare them with the predetermined reference dataset, and update the brain stimulation protocol dynamically so as to optimise the brain stimuli in maintaining balance between the sympathetic division and the parasympathetic division of the nervous system. Examples of the at least one adaptive learning algorithms may include, but are not limited to, reinforcement learning, neural network models, or regression models. Moreover, the term “computational algorithm” refers to a set of rule-based or mathematical instructions configured to process the one or more signal features and map them to the one or more stimulation parameters of the brain stimulation protocol.

Herein, the at least one adaptive learning algorithm dynamically adjusts the one or more stimulation parameters in response to deviations one or more signal features or the physiological markers such as HRV, thereby creating the closed-loop feedback mechanism. Moreover, the at least one computational algorithm may apply deterministic operations, such as comparing one or more signal features against a threshold or calculating LF/HF ratios, to select one or more stimulation parameters directly. Furthermore, the processing unit uses the at least one adaptive learning algorithm and/or the at least one computational algorithm to generate or update the brain stimulation protocol, which is then communicated to the input/output arrangement for execution via the plurality of electrodes. Moreover, it provides flexibility in achieving real-time, personalised, and reliable modulation of brain activity. The at least one adaptive learning algorithm ensures that the wearable device can handle inter-individual differences and temporal variations in the user's brain and physiological signals, while the at least one computational algorithm provide fast, stable, and interpretable results for safety and consistency. Optionally, the information of the set is derived using the adaptive learning algorithm or the computational algorithm.

A technical effect of the at least one processing algorithm comprising at least one of: the at least one adaptive learning algorithm, the at least one computational algorithm.is that the wearable device enables the closed-loop operation of the brain stimulation protocol and enhances the effectiveness of the brain stimulation protocol, reduces the risk of overstimulation or inefficacy, and ensures that the brain stimuli that has been applied at the mastoid region, reflects both real-time physiological changes and stable computational baselines. As a result, the wearable device provides improved therapeutic accuracy, user safety, and adaptability across the one or more physiological markers and user profiles.

Optionally, the at least one adaptive learning algorithm comprises at least one of: a K-nearest neighbour algorithm, a regression analysis, an ensemble tree-based algorithm, maximum power point tracking, an artificial neural network, a deep convolutional neural network, a recurrent neural network, a reinforcement learning algorithm, a random forest algorithm, a recommender system, genetic algorithm, a Q-learning algorithm, a deep Q-learning algorithm, and a Bayesian optimisation algorithm

A technical effect of the aforementioned feature is that the wearable device achieves enhanced adaptability and precision in updating the brain stimulation protocol. Moreover, a diversity of at least one adaptive algorithm allows the processing unit to model complex, non-linear relationships in the one or more signal features and the one or more physiological markers, to predict user-specific responses, and to iteratively refine one or more stimulation parameters. Consequently, the brain stimuli generated and applied via the input/output arrangement are personalised in the real time, thereby improving therapeutic accuracy, reducing the risk of overstimulation or inefficacy, and ensuring stable modulation of brain activity to maintain balance between the sympathetic division and the parasympathetic division of the nervous system. Herein, the term “K-nearest neighbour algorithm” classifies or predicts the one or more stimulation parameters by comparing the one or more signal features with the most similar examples in the predetermined reference data set. The term “regression analysis” models the relationship between the one or more signal features and the one or more physiological markers, to compute continuous values for one or more stimulation parameters. The term “ensemble tree-based algorithms” combine multiple decision trees to identify pattern in the one or more signal features and accordingly, select the one or more stimulation parameters improving accuracy and reducing variability in the brain stimulation protocol. The term “maximum power point tracking” refers to the at least one adaptive learning algorithm which dynamically adjusts one or more stimulation parameters in the brain stimulation protocol to maintain an efficient application of the brain stimuli on the mastoid region of the user under one or more physiological markers. The term “artificial neural network” refers to a computation model which maps complex, non-linear relationships between one or more signal features and one or more physiological markers, to generate personalised one or more stimulation parameters. The term, “deep convolutional neural network” refers to an algorithm which extracts hierarchical patterns from one or more signal features to refine one or more stimulation parameters for the application of the brain stimuli. The term “recurrent neural network” refers to the at least one adaptive algorithm that processes the one or more signal features over time to predict variations in one or more physiological markers, enabling adaptive adjustment of the one or more stimulation parameters. The term “reinforcement learning algorithm” refers to the at least one adaptive algorithm that iteratively updates the one or more stimulation parameters in the brain stimulation protocol based on feedback from the one or more physiological markers. The term “random forest algorithm” refers to the at least one adaptive algorithm which applies multiple decision trees to one or more signal features of the one or more stimulation parameters, thereby selecting robust said one or more stimulation parameters for the brain stimulation protocol. The term “recommender system” refers to the at least one adaptive algorithm that predicts and suggests one or more stimulation parameters by comparing the one or more signal features and one or more physiological markers of the user from the predefined reference data sets. The term “genetic algorithm” refers to the at least one adaptive algorithm that evolves the one or more stimulation parameters by simulating natural selection processes, progressively optimising the brain stimulation protocol. The term “Q-learning” refers to the at least one adaptive algorithm which determines a mapping between one or more signal features and adjusts the one or more stimulation parameters by maximising a reward function tied to improvements in one or more physiological markers. The term “deep Q-learning algorithm” extends the Q-learning with a deep neural network to handle high dimension one or more signal features, enabling precise selection of the one or more stimulation parameters in complex physiological state of the user. All the aforementioned types of at least one adaptive learning algorithm are well-known in the art.

Optionally, the wearable device comprises a headset. In the regard, the term “headset” refers to a form of the headwear arrangement of the wearable device that is configured to be worn on the head of the user and comprises one or more supporting structures, such as an earcup, a headband, or an earpiece, that house and support the plurality of electrodes. Optionally, the headset includes the input/output arrangement, the data processing arrangement, one or more power units. In an exemplary implementation, the headset is a wireless headphone. A technical effect of the aforementioned feature is that the wearable device becomes more ergonomic, unobtrusive, and familiar to the user, thereby improving long-term wearability and user compliance. Moreover, incorporating the electrode arrangement into a headset form factor allows the plurality of electrodes to be consistently positioned over the mastoid region without requiring precise alignment by the user, ensuring stable electrical contact during both monitoring and stimulation.

Optionally, the plurality of electrodes are flexible, said plurality of electrodes being configured to adapt to a contour of the skin of the user, when the headwear arrangement is worn over the head of the user. In this regard, the plurality of electrodes is fabricated using materials which are flexible, biocompatible and electrically conductive. Examples of the material may include, but are not limited to, conductive polymers, graphene composites, conductive fabrics, or thin metallic films deposited on elastic substrates. These materials allow the plurality of electrodes to adapt to uneven surfaces contours of the mastoid region and maintain uniform electrical contact with the skin of the user, even during user movement. Optionally, the plurality electrodes are embedded in or supported by the flexible cushioning member of the headwear arrangement, which enhances comfort and ensures reliable electrode-skin coupling without excessive pressure.

A technical effect of flexibility of the plurality of electrodes is that it improves a signal acquisition quality and the reliability of the generated brain stimulation protocol. Moreover, by adapting to the contour of the skin, the plurality of electrodes reduce impedance variability, minimise artefacts, and maintain stable contact with the skin of the user during extended wear which leads to more accurate monitoring of brain activity, more effective delivery of brain stimuli to the at least partially in the mastoid region of the user, enhanced user comfort, and improved long-term compliance, thereby increasing the overall efficacy and usability of the wearable device in real-world conditions.

Optionally, the plurality of electrodes are arranged to collectively cover a surface area of the skin of the user, lying in a range of 500 millimetres squared to 2500 millimetres squared. As an example, the plurality of electrodes are arranged to collectively cover the surface area of the skin of the user lying in the range from 500, 550, 600, 700, 850, 1000, 1200, 1450, 1700, or 2000 millimetres squared up to 1000, 1050, 1100, 1200, 1350, 1500, 1700, 1950, 2200, or 2500 millimetres squared. This range is selected to ensure that the plurality of electrodes collectively cover the surface area of the skin of the user, particularly the mastoid region, to achieve both reliable acquisition of the electrical signals corresponding to the brain activity of the user and effective delivery of the brain stimuli.

A technical effect of the aforementioned feature is that the wearable device ensures a stable and uniform electrical contact with the skin of the user. The range covered by the plurality of electrodes is sufficient to maintain low electrode-skin impedance, improves signal-to-noise ratio of the electrical signals corresponding to the brain activity of the user, and enable effective distribution of the brain stimuli across the mastoid region.

Optionally, the flexible cushioning member comprises any one of: a silicon pad, a foam pad. In this regard, the term “silicon pad” refers to a cushioning element formed from silicone-based elastomeric material that is flexible, biocompatible, electrically insulating, and capable of maintaining its shape under repeated mechanical stress. The silicon pad is configured to distribute pressure uniformly across the plurality of electrodes, thereby improving comfort and maintaining consistent contact of the plurality of electrodes with the skin of the user. Moreover, the term “foam pad” refers to a cushioning element fabricated from flexible, compressible foam material, such as polyurethane or memory foam, that conforms to the contour of the skin of the user when the headwear arrangement is worn. The foam pad provides both mechanical cushioning and positional stability for the plurality of electrodes while allowing air circulation and reducing irritation during extended use. A technical effect of the aforementioned feature is that improves the comfort, stability, and reliability of the contact between the plurality of electrodes with the skin of the user. Moreover, the flexible cushioning member allows greater user compliance during extended wear, improved safety of the wearable device, and more reliable execution of the brain stimulation protocol under real-world conditions.

Optionally, the flexible cushioning member comprises a conductive solution to provide electrical coupling between the plurality of electrodes and the skin of the user. In this regard, the term “conductive solution” refers to a liquid or gel-based medium that is electrically conductive and biocompatible, configured to enhance the coupling between the plurality of electrodes and the skin of the user. Examples of conductive solutions may include, but are not limited to, saline solutions, electrolyte gels, hydrogels, or polymeric ionic liquids. The conductive solution provides a low-resistance path for the transfer of electrical signals, thereby reducing impedance at the coupling between the plurality of electrodes and the skin of the user. Herein, the conductive solution fills micro-gaps and irregularities at the contact between the plurality of electrodes and the skin of the user, ensuring consistent surface contact across the mastoid region when the headwear arrangement is worn. A technical effect of using the conductive solution to provide the electrical coupling between the plurality of electrodes and the skin of the user is that it improves the signal-to-noise ratio of the electrical signals and enhances the consistency of the applied brain stimuli. Moreover, by using the conductive gel lowering impedance, reducing artefacts, and stabilising the contact between the plurality of the electrodes and skin of the user even during extended wear or user movement ensuring that the input/output arrangement reliably acquires high-quality electrical signals while effectively applying brain stimuli to the at least partially in the mastoid region of the user.

Optionally, the flexible cushioning member comprises a porous electrolyte carrier saturated with saline, the porous electrolyte carrier being configured to maintain electrical contact between each of the plurality of electrodes and the skin of the user. In this regard, direct dry contact between the plurality of electrodes and the skin results in high impedance, unstable coupling, and skin irritation during the extended use of the wearable device. Therefore, incorporating the porous electrolyte carrier saturated with saline improves the compatibility of the contact between the plurality of electrodes and the skin of the user along with reduces skin irritation while ensuring effective electrical coupling. In an implementation, the porous electrolyte carrier, typically, a sponge material, is impregnated with an electrolyte solution such as physiological saline solution (approximately 150 millimolar sodium chloride solution). Herein, the porous electrolyte carrier conforms to the contour of the skin and provides a moist, conductive interface that ensures stable coupling between the plurality of electrodes and the skin of the user. Moreover, the porous electrolyte carrier creates a uniform conductive pathway for both receiving electrical signals corresponding to the brain activity of the user and delivering the brain stimuli according to the brain stimulation protocol. A technical effect of the aforementioned feature is that it reduces skin irritation, lower impedance and maintains reliable electrical contact between the plurality of electrodes and the skin of the user even during prolonged wear. Consequently, the wearable device achieves safer and more effective execution of the brain stimulation protocol in real-world conditions.

Optionally, the brain stimuli are delivered as a current signal having an amplitude that lies in a range of 0.1 milliAmpere to 2 milliAmperes. As an example, the brain stimuli are delivered as the current signal having the amplitude that lies in a range of 0.1, 0.5, 0.7, 1, 1.3, or 1.5 milliAmperes up to 1, 1.2, 1.5, 1.8, or 2 milliAmperes. This range is selected because it ensures reliable application of the brain stimuli to the skin lying at least partially in the mastoid region, thereby improving the accuracy and consistency of the applied brain stimulation protocol, reducing the risk of skin irritation or unintended tissue activation, and enhancing user compliance during extended operation. A technical effect of the aforementioned feature is that it allows the wearable device to apply the brain stimulation protocol that is effective for modulating the brain activity of the user while remaining safe and comfortable at the skin of the user.

Optionally, a control unit is communicably coupled with the data processing arrangement, wherein, when in operation, the control unit is configured to:
receive input from at least one user device; and
transmit the input to the processing unit of the data processing arrangement,
   wherein the processing unit of the data processing arrangement is further configured to:
receive the input from the control unit;
update the brain stimulation protocol to generate an updated brain stimulation protocol, based on the received input; and
transmit the updated brain stimulation protocol to the input/output arrangement, and
   wherein the control unit comprises a communication module that is configured to establish a communication link between the processing unit and a processor of the at least one user device.

In this regard, “control unit” refers to a programmable and/or non-programmable arrangement that is communicably coupled with the data processing arrangement and is configured to receive input from the at least one user device and transmit the input to the processing unit of the data processing arrangement. Optionally, the control unit comprises a user interface such as a button interface, a touchscreen interface, a wireless interface, a gesture interface, a microphone interface (voice detection), or other suitable means for capturing instructions from the user. Moreover, the term “user device” refers to a computing or electronic device that is communicably coupled with the control unit via the communication module. The user device can be the device of the user who is wearing or operating the wearable device. Examples of the user device may include, but are not limited to a smartphone, a smartwatch, a tablet, a personal computer running a dedicated application, or a third-party user device such as a clinician console, a cloud server, or a remote monitoring system. Optionally, the user device is configured to provide the input to the control unit in the form of operational parameters including, but not limited to, an ON/OFF state, a stimulation mode, a stimulation duration, a medical history of the user, or a desired mental state of the user. Furthermore, the term “communication module” refers to a component of the control unit that establishes a wired communication link or wireless communication link between the processing unit of the data processing arrangement and a processor of the at least one user device. Examples of the communication module may include, but are not limited to, the Internet, Wi-Fi, Bluetooth®, mobile networks, and local wired connections.

Herein, the control unit receives input from the at least one user device and transmits the input to the processing unit of the data processing arrangement. The processing unit, upon receiving the input, updates the brain stimulation protocol. Subsequently, the brain stimulation protocol is transmitted by the processing unit to the input/output arrangement. Consequently, the input/output arrangement generate and apply the brain stimuli protocol. Examples of the input may include, but are not limited to, a stimulation mode, a duration, or user-specific preferences.

In an exemplary implementation, the at least one user device may provide input related to the stimulation mode. Subsequently, the control unit may relay the input to the processing unit. Moreover, the processing unit then modifies the brain stimulation protocol by adjusting the one or more stimulation parameters, allowing the brain stimuli generated by the input/output arrangement to be adapted dynamically according to the user specific preferences or clinical requirements. Optionally, the at least one user device is implemented as an application for iOS® or Android® operating systems, wherein an application provides an interface for the user to configure one or more stimulation parameters and transmit the same to the control unit for updating the brain stimulation protocol.

A technical effect of the aforementioned feature is that it updates the brain stimulation protocol in real time based on received input resulting in improved adaptability of the wearable device, ensuring that the brain stimuli are optimized to the user's current physiological and therapeutic needs, and allows integration of clinician-directed adjustments or algorithmic updates.

Optionally, an external stimulation arrangement for providing at least one of: a visual stimulation, an audio stimulation, a virtual reality stimulation, concurrently with the delivery of the brain stimuli to the at least one of the plurality of electrodes, the external stimulation arrangement being communicably coupled with the control unit.

In this regard, the term “external stimulation arrangement” refers to an external device used for providing extra stimulation along with the brain stimuli. The external stimulation arrangement comprises one or more devices, wherein the one or more devices comprises at least one of: a virtual reality device, a virtual reality headset, a display device, a pair of glasses, a pair of smart glasses, headphones, earphones, a speaker, a therapeutic massager, electrodes placed elsewhere on the body and/or a smart lens. The external stimulation arrangement is communicably coupled with the data processing arrangement. Moreover, the external stimulation arrangement is configured to receive electrical power from one or more power units.

Herein, the data processing arrangement generates the brain stimulation protocol comprising the one or more stimulation parameters. The one or more stimulation parameters are synchronised with the electrical signals which are then transmitted to the external stimulation arrangement, coordination with the brain stimuli applied to the mastoid region of the user.

Moreover, the external stimulation arrangement providing the at least one of: the visual stimulation, the audio stimulation, the virtual reality stimulation helps the user to relax, which brings down stress level when operated in synchronisation with the brain stimuli. Advantageously, the external stimulation arrangement provides isolation to the user by reducing any unwanted light coming to the eyes of the user and any noise coming to the ears of the user. Such an isolation helps the user to further reduce unwanted brain activity, resulting in enhanced effectiveness of the brain stimulation protocol.

Optionally, the input/output arrangement further comprises an acoustic output arrangement that is operably coupled with the data processing arrangement, wherein the data processing arrangement is configured to deliver the acoustic output, via the acoustic output arrangement concurrently with the delivery of the brain stimuli to the at least one of the plurality of electrodes. The concurrent delivery of audio, visual, or virtual reality stimulation with the brain stimuli amplifies the modulation of brain activity by reinforcing desired neural responses. The acoustic output arrangement further strengthens the effect by providing a built-in pathway for delivering auditory signals directly through the input/output arrangement, ensuring synchronisation and reducing reliance on separate external hardware.

A technical effect of the aforementioned feature is external stimulation arrangement for providing the at least one of: the visual stimulation, the audio stimulation, the virtual reality stimulation, concurrently with the delivery of the brain stimuli is that provides multimodal stimulation that enhances relaxation, reduces stress, minimises unwanted sensory input, and promotes improved engagement of the user. Consequently, the wearable device achieves higher therapeutic efficacy, greater comfort, and expanded versatility in both personal and clinical use.

Optionally, the input/output arrangement comprises a safety arrangement configured to disable applying the brain stimuli to the plurality of electrodes and receiving the electrical signals from the plurality of electrodes, upon detection of at least one abnormal operating condition. Herein, the term “safety arrangement” refers to a component or set of components included in the input/output arrangement, which is configured to monitor the operation of the wearable device and to disable both the application of the brain stimuli to the plurality of electrodes and the receiving of the electrical signals from the plurality of electrodes, upon detection of at least one abnormal operating condition. The safety arrangement may be implemented in hardware, software, firmware, or any combination thereof. Examples of the safety arrangements may include, but not limited to, monitoring circuits, watchdog timers, diagnostic algorithms, impedance monitoring modules. Moreover, the term “abnormal operating condition” refers to an operational state in which continued functioning of the wearable device poses a potential risk to the user. The at least one abnormal operating condition comprises at least one of: hardware faults, software crashes, unsafe stimulation parameters, electrode detachment, impedance spikes. The safety arrangement ensures safe operation of the wearable device under varying user conditions and environments. The wearable device operates in close contact with the skin of the user and targets sensitive neural pathways. Therefore, by incorporating the safety arrangement, the wearable device ensures that the application of the brain stimuli is halted immediately under at least one abnormal operating condition, so as to prevent an unintended application of the brain stimuli to the plurality of electrodes and to ensure safe operation of the wearable device. A technical effect of the safety arrangement comprised in the input/output arrangement is that it provides enhanced protection from any damage to the user in a real-time manner resulting risk-free usage of the wearable device without requiring expert assistance. Moreover, the wearable device is designed in its external and internal component parts, and also in its manner of operation, such that any occurrence of harm to the user is avoided, while ensuring safe, reliable, and effective execution of the brain stimulation protocol.

The present disclosure also relates to the first aspect as described above. Various embodiments and variants disclosed above, with respect to the aforementioned first aspect, apply mutatis mutandis to the second aspect.

Optionally, the data processing arrangement further comprises a memory module that stores the predetermined reference data set, the method further comprising iteratively updating the predetermined reference dataset in the memory module, when applying the at least processing algorithm.

Optionally, the method further comprises monitoring one or more physiological markers corresponding to the brain stimuli, the one or more physiological markers comprising at least one of: a heart rate variability parameter, a beat-to-beat interval, a ratio of low frequency to high frequency.

### Experimental Part

In the experiment that was conducted, 62 healthy volunteers with ages lying in the range of 18 to 49 years were recruited. The 62 healthy volunteers were divided into three groups, namely, a Stimulation group, a Sham group, and an Excluded group. In the Stimulation group, there were 26 volunteers, wherein a mean of their ages was 26.3, a standard deviation of their ages was 7.81 years. In the Sham group, there were 22 volunteers, wherein a mean of their ages was 26.1, a standard deviation of their ages was 6.47 years. In the Excluded group, there were 14 persons that were excluded for different reasons. The remaining 48 participants (24 employees, telecom operators of a contact centre of telecommunication company (6 males and 18 females), 24 students and employees of an university (5 males and 19 females) were randomly divided into two equivalent study groups (the Stimulation group and the Sham group) in a double-blinded way.

The volunteers were eligible to enroll in the study if they were over 18 years old and did not have clinical manifestations of mental disorders or cognitive impairment, neurological, cardiovascular diseases, problems related to the vestibular system and were not taking psychoactive medication, drugs, or alcohol. Additional exclusion criteria were skin diseases, wounds and cracks at the stimulation site, metal implants in the head or piercings under the stimulation site. Moreover, the volunteers were asked to abstain from alcohol for at least 12 hours before the stimulation session.

The study was carried out according to the protocol in 6 sessions spread over a period of approximately two weeks. Moreover, psychological testing was carried out on the first (“0”) and last (“4+1”) days of the study. Furthermore, the state of autonomic regulation of the cardiovascular system (i.e., heart rate variability (HRV)) was measured continuously on each day the stimulation was administered as well as the first and the last days of the study. Stimulation was carried out on 4 days with at least a day between the stimulation sessions for 6 min, with the exact protocol delivered (tVNS or Sham) dependent on the randomisation group. Neither the participant, nor the experimenter present were aware of the protocol being delivered.

The Stimulation/SHAM was arranged as a 4-day course of 6-minutes stimulation sessions using the wearable device. The wearable device was a combination of wireless headphones that broadcast music or relaxing high quality sounds, and wireless non-invasive current stimulators (micropolarizers) with safe parameters of the generated electric current. The current stimulator (electrodes built into the headphones) consists of an analog-to-digital converter and a circuit that allows the delivery of a weak current signal (current up to +/−1.5 mA and voltage up to ˜+/−30 V) to the electrodes. Since the amplitude utilised in this study was limited by the “LOW” setting, the maximum delivered current was ˜0.6 mA, that is, peak-to-peak wave amplitude was ˜1.2 mA. For the safety of stimulation, in addition to the limitations included in the application, the electronics itself contain 2 mA hardware limits in both directions. A porous electrolyte carrier (i.e., a sponge) saturated with electrolyte (˜150 mM NaCl) is used as an interface between the electronics and the skin, which reduces skin irritation.

A software application was used by the experimenter, that was controlled by a cloud-based system for randomisation and for delivery of assigned brain stimulation protocols to the wearable device as well as for collection of data on the execution of the brain stimulation protocol. This experiment used a brain stimulation protocol that combined pleasant meditative sounds and slow bipolar wave (0.1 Hz) electrical stimulation, to cause a relaxing effect and, according to preliminary results, may increase cognitive abilities. The sham stimulation consisted of 10s 0.1 Hz, stimulation at 0.6 mA, to provide a false sense of real stimulation, after which the maximum delivered placebo stimulation current was ˜0.05 mA, thus peak-to-peak wave amplitude was ˜0.03-0.06 mA.

In this experiment, the stimulation zone included the mastoid area where the auricular branch of the vagus nerve exits the cranium by passing through the tympanomastoid fissure between the mastoid process and the tympanic part of the temporal bone, and divides into two branches (first one joins the posterior auricular nerve, the other spreads to the skin of the auricle area of the ear and to the posterior part of the ear canal). Below the mastoid processes, the vestibular nerve directs from the inner ear to the vestibular nuclei of the brain stem, which, in turn, are interconnected with the relay nuclei of the thalamus (ventroposterolateral thalamic nucleus).

Moreover, heart rate monitoring was carried out throughout the entire day of the experiment by a chest strapped heart rate monitor, and physiological indicators were processed. Each heart rate record was pre-processed for artefact removal, divided into time-stamped 6-minute samples. The 6-minute heart rate samples (pre-stimulation (baseline), stimulation, and post-stimulation condition) were processed with the calculation of 88 indicators (HRV time-domain, frequency-domain, and non-linear measures).

The used HRV time-domain indicators are reflecting the balance of activity of sympathetic and parasympathetic divisions of the autonomic nervous system. Moreover, RMSSD (ms) is used to assess vagal-mediated changes reflected in HRV and parasympathetic activity and is calculated as root-means-square difference between successive RR intervals.

In order to measure a severity of emotional burnout in students, 22-item Maslach Burnout Inventory (MBI) was used. The MBI is designed for the diagnosis of occupational burnout and comprises of three components: emotional exhaustion (EE, the 9-item scale), depersonalization (DP, the 5-item scale) and reduction of personal achievements (PA, the 8-item scale)

Beneficial changes in the psychoemotional state of the respondents were revealed after the stimulation course. The Positive and Negative Affect Assessment (PANAS) indicates a positive effect of stimulation on improving mood (p=0.027; F=2.588). There are detected significant reductions in the severity of anxiety (GAD-7, p=0.028; F=5.551) and indicators of occupational stress (PSM-25, p=0.007; F=8.677). Unexpectedly, depression scores also revealed an improvement (IDS, p=0.014; F=6.471). A set of stimulation significantly improved the job burnout (MBI, reduction of personal achievements, p<0.001; F=20.893). In combination, the data appears to corroborate that the stimulation attenuates negative impact of job-related stress and improves emotional state associated with professional activities.

A significant increase in RMSSD (F(2, 64)=5.76 p=0.011) at the time of stimulation compared to the control group indicates an activating effect on the parasympathetic division of the autonomic nervous system. In contrast, no significant differences were found in the SHAM group. The effect of stimulation turned out to be short-term, which was manifested in a drop in the value of the RMSSD parameter immediately after the end of stimulation.

Moreover, an increase in the vagus nerve tone was reflected in the changes in the spectral power of high-frequency (HF)—there was an increase in the absolute power of HF bands (HF, F(2, 64)=4.28, p=0.032) and the relative value of the power of HF bands (HF %, F(2, 64)=5.30, p=0.021), relative value of HF band power, expressed in normalized units (HF in n.u., F(2, 64)=5.35, p=0.019).

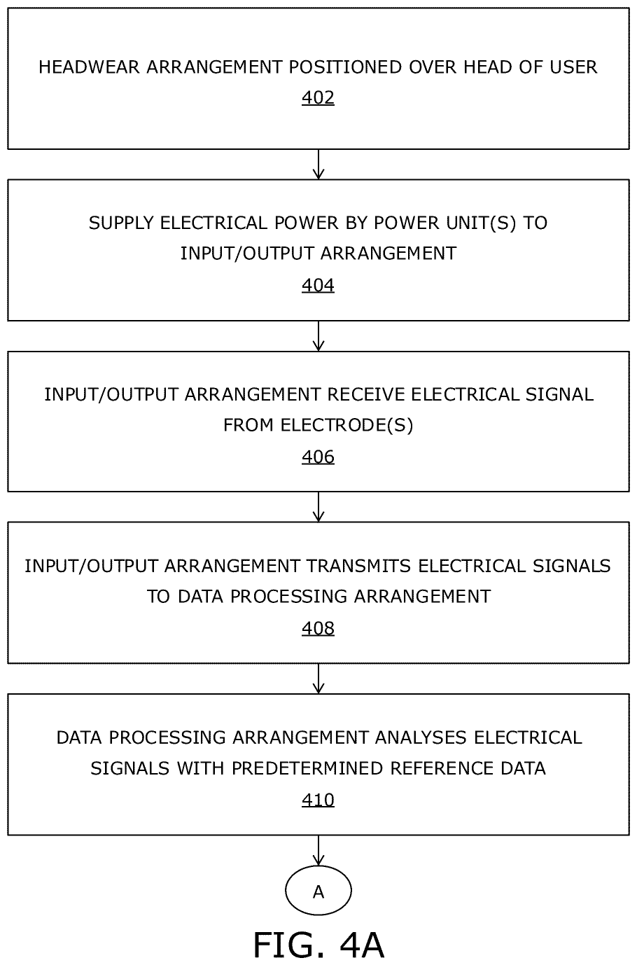

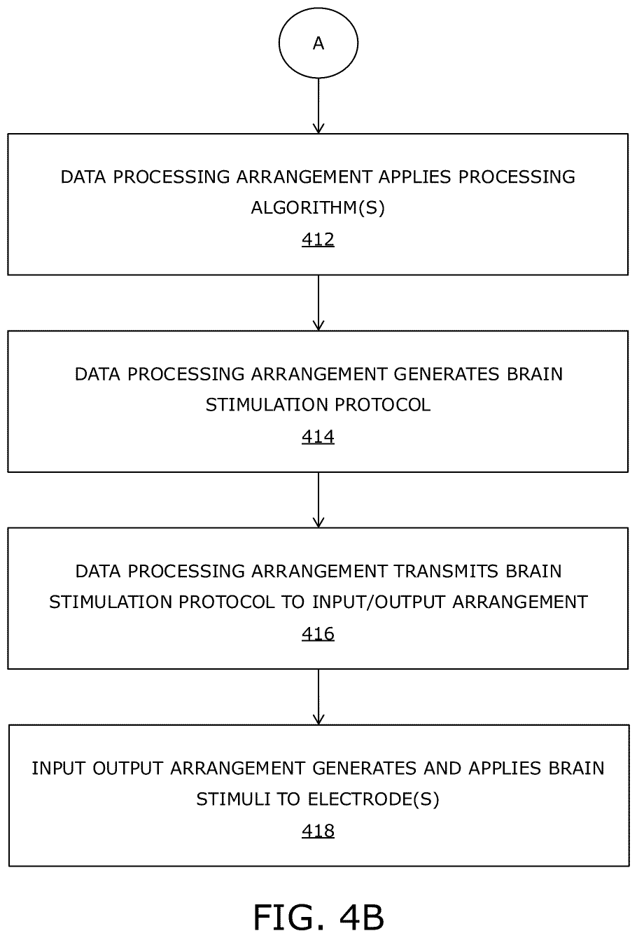

The vagosympathetic balance coefficient showed a statistically significant decrease in the stimulation group at the time of stimulation (FIG. 4): LF/HF indicator (F(2, 64)=3.16, p=0.048), LF/HF_AR indicator (F(2, 64)=3, 91, p=0.03). This reflects a shift towards parasympathetic nervous system dominance.

Thus, the obtained data indicates that positive effects of used stimulation technique are realized by vagus nerve through central mechanisms.

### Conclusion

The obtained data detected the activation of the parasympathetic nervous system in response to the brain stimulation protocol, which is characteristic of the resting state. An increase in the parasympathetic nervous system activation indicates a high degree of recovery and a capacity for handling stress. The observed positive dynamics of improvement in indicators of stress, anxiety, depression, professional burnout and mood is of particular importance as it indicates the high effectiveness of the method used to stimulate the vagus nerve. Taken together with the protocol's reasonable safety profile and the fact that this experiment was performed in office and academic environments, the results of this study suggest that the brain stimulation protocol has the potential to become an effective tool for mental wellbeing for work and for education environments.

### DETAILED DESCRIPTION OF THE DRAWINGS

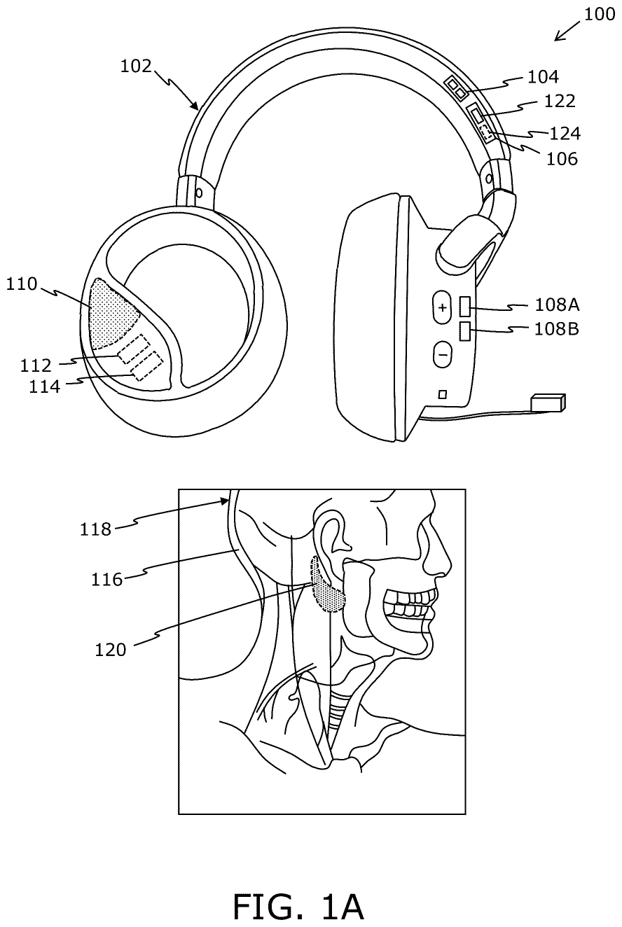

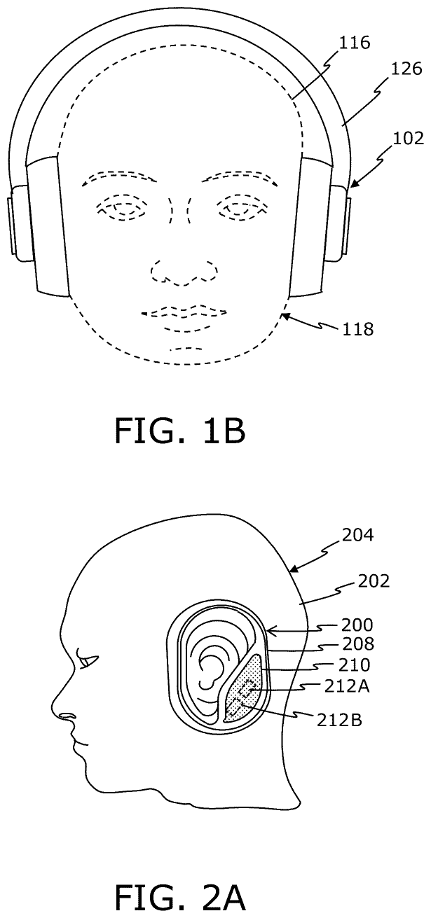

Referring to FIG. 1A, illustrated is a schematic illustration of a wearable device 100 for monitoring brain activity and for providing non-invasive stimulation, and referring to FIG. 1B, illustrated is an exemplary implementation of a headwear arrangement 102 in use, in accordance with an embodiment of the present disclosure. With reference to FIG. 1A, the wearable device 100 comprises a headwear arrangement 102, an input/output arrangement 104, a data processing arrangement 106, and one or more power units (depicted as two power units 108A and 108B). Herein, the headwear arrangement 102 comprises an electrode arrangement (not shown), and a flexible cushioning member 110. The electrode arrangement comprises a plurality of electrodes (depicted as two electrodes 112 and 114). Herein, when the headwear arrangement 102 arrangement is worn over a head 116 of a user 118, the two electrodes 112 and 114 are configured to make electrical contact with a skin of a user 118. The skin is lying at least partially in a mastoid region 120 of the user 118. Optionally, the headwear arrangement 102 is implemented as a headset. The flexible cushioning member 110 supports the two electrodes 112 and 114. Herein, the flexible cushioning member 110 maintains a physical contact between the two electrodes 112 and 114 and the skin of the user 118. The input/output arrangement 104 is operably coupled with the two electrodes 112 and 114. Herein, when in operation, the input/output arrangement 104 is configured to: receive electrical signals from at least one of the two electrodes 112 and 114, wherein the electrical signals correspond to the brain activity of the user 118. Moreover, the input/output arrangement 104 is configured to: transmit the electrical signals to the data processing arrangement 106 to process the electrical signals for generating brain stimuli, based on a brain stimulation protocol. Furthermore, the input/output arrangement 104 is configured to generate and apply the brain stimuli to the at least one of the two electrodes 112 and 114. The brain stimuli are generated using the brain stimulation protocol received processing arrangement 106, wherein the brain stimuli are applied on the skin lying at least partially in the mastoid region 120. The data processing arrangement 106 is communicably coupled with the input/output arrangement 104, wherein the data processing unit 106 comprises a processing unit 122. Herein, the processing unit 122 is configured to perform various operations, as described earlier with respect to the aforementioned first aspect. The two power units 108A and 108B is electrically coupled with the input/output arrangement 104 and the data processing arrangement 106. The two power units 108A and 108B supplies electrical power to the input/output arrangement 104 and the data processing arrangement 106. Optionally, the data processing arrangement 106 further comprises a memory module 124 stores a predetermined reference data set. Herein, the processing unit 122 is further configured to iteratively update a predetermined reference dataset in the memory module 124, when applying an at one least processing algorithm.

With reference to FIG. 1B, there is shown the headwear arrangement 102 in use. Herein, the headwear arrangement 102 is worn over the head 116 of the user 118 covering at least partially the mastoid region (not shown) of the user 118.

FIGS. 1A and 1B are merely examples, which should not unduly limit the scope of the claims herein. The person skilled in the art will recognize many variations, alternatives, and modifications of embodiments of the present disclosure.

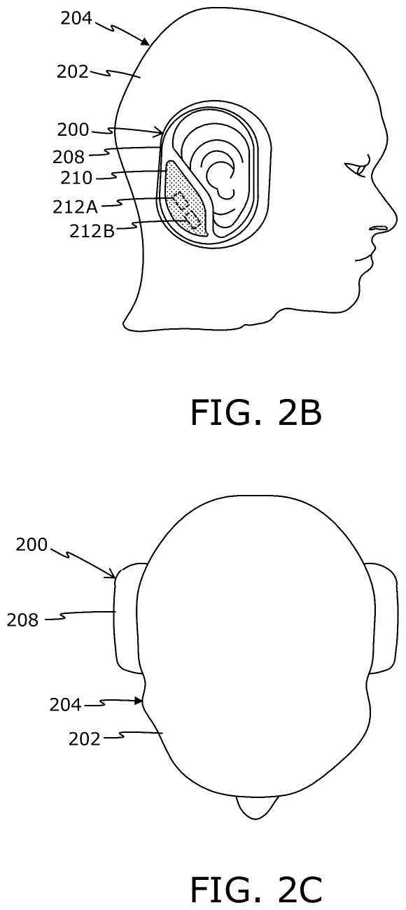

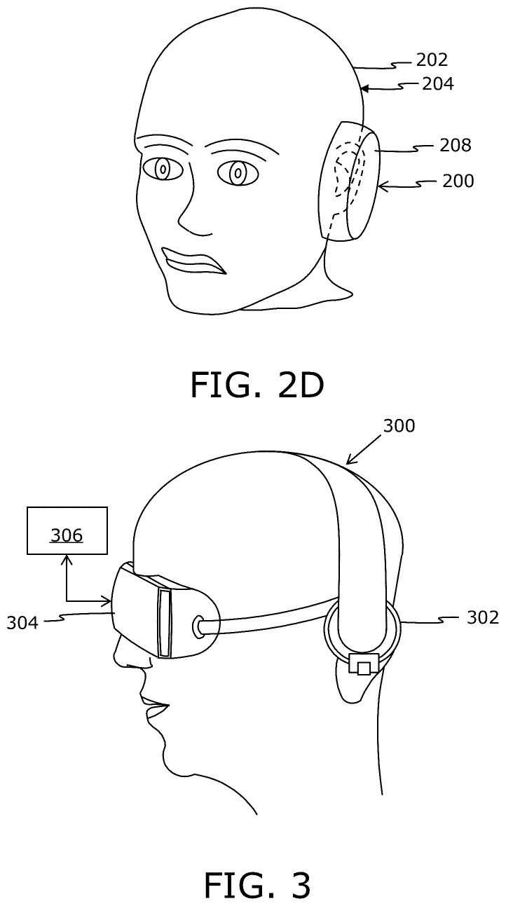

Referring to FIGS. 2A, 2B, 2C, and 2D collectively, there are illustrated different perspective views of placement of a portion of a wearable device 200 over head 202 of a user 204, in accordance with an embodiment of the present disclosure. In FIGS. 2A and 2B, the portion of the wearable device 200 comprises a headwear arrangement 206. The headwear arrangement 206 comprises an electrode arrangement (not shown) and a flexible cushioning member 208. The electrode arrangement comprises a plurality of electrodes (depicted as two electrodes 210A and 210B) wherein, the two electrodes 210A and 210B are configured to make electrical contact with a skin of the user 204. Moreover, the skin lying at least partially in a mastoid region (not shown) of the user 204. The flexible cushioning member 210 supports the two electrodes 210A and 210B. Herein, the flexible cushioning member 208 maintains a physical contact between the two electrodes 210A and 210B and the skin of the user 204. With reference to FIG. 2A, there is shown a left profile view of the user 204. With reference to FIG. 2B, there shown a right profile view of the user 204. With reference to FIG. 2C, there is shown a top view of the user 204. With reference to FIG. 2D, there is shown a perspective view of the user 204.

FIGS. 2A, 2B and 2C are merely examples, which should not unduly limit the scope of the claims herein. The person skilled in the art will recognize many variations, alternatives, and modifications of embodiments of the present disclosure.

Referring to FIG. 3, illustrated is an exemplary implementation of a wearable device 300 for monitoring brain activity and for providing non-invasive stimulation, in accordance with an embodiment of the present disclosure. Herein, the wearable device 300 comprises the headwear arrangement 302 and an external stimulation arrangement 304. The external stimulation arrangement 304 provides at least one of: a visual stimulation, an audio stimulation, a virtual reality stimulation. The external stimulation arrangement is communicably coupled with a control unit 306.

FIG. 3 is merely an example, which should not unduly limit the scope of the claims herein. The person skilled in the art will recognize many variations, alternatives, and modifications of embodiments of the present disclosure.

Referring to FIGS. 4A and 4B collectively, illustrated are steps of a method for monitoring a brain activity and providing non-invasive stimulation, the method being implemented by a wearable device, in accordance with an embodiment of the present disclosure. At step 402, a headwear arrangement is positioned over a head of a user. The headwear arrangement comprising a plurality of electrodes configured to make electrical contact with skin of the user lying at least partially in a mastoid region of the user. At step 404, electrical power is supplied by one or more power units, to an input/output arrangement and a data processing arrangement. At step 406, the input/output arrangement receives electrical signals from at least one of the plurality of electrodes. The input/output arrangement that is operably coupled with the plurality of electrodes. Herein, the electrical signals correspond to the brain activity of the user. At step 408, the input/output arrangement transmits the electrical signals to the data processing arrangement. At step 410, the data processing arrangement analyses the electrical signals with a predetermined reference data set by extracting one or more signal features. At step 412, the data processing arrangement applies at least one processing algorithm to map the one or more signal features to one or more stimulation parameters to provide a balance between a sympathetic division and a parasympathetic division of a nervous system. At step 414, the data processing arrangement generates a brain stimulation protocol comprising the one or more stimulation parameters. At step 416, the data processing arrangement transmits the brain stimulation protocol to the input/output arrangement. At step 418, the input/output arrangement generates and applies the brain stimuli to the at least one of the plurality of electrodes using the brain stimulation protocol, the brain stimuli being applied on the skin lying at least partially in the mastoid region.

The aforementioned steps are only illustrative and other alternatives can also be provided where one or more steps are added, one or more steps are removed, or one or more steps are provided in a different sequence without departing from the scope of the claims herein.

1. A wearable device for monitoring brain activity and for providing non-invasive stimulation, the wearable device comprising:
a headwear arrangement comprising an electrode arrangement having a plurality of electrodes, wherein when the headwear arrangement is worn over a head of a user, the plurality of electrodes are configured to make electrical contact with a skin of a user, the skin lying at least partially in a mastoid region of the user;
an input/output arrangement that is operably coupled with the plurality of electrodes, wherein, when in operation, the input/output arrangement is configured to:
receive electrical signals from at least one of the plurality of electrodes, wherein the electrical signals correspond to at least one of: brain activity of the user, heart activity of the user, vagus nerve activity of the user;
transmit the electrical signals to a data processing arrangement to process the electrical signals for the purpose of generating brain stimuli, based on a brain stimulation protocol; and
generate and apply the brain stimuli to the at least one of the plurality of electrodes, the brain stimuli being generated using the brain stimulation protocol received from the data processing arrangement, wherein the brain stimuli are applied on the skin lying at least partially in the mastoid region;
the data processing arrangement communicably coupled with the input/output arrangement, the data processing arrangement comprising a processing unit, wherein the processing unit is configured to:
receive the electrical signals from the input/output arrangement;
analyse the electrical signals;
apply at least one processing algorithm to map one or more signal features to one or more stimulation parameters in the brain stimulation protocol;
generate the brain stimulation protocol comprising the one or more stimulation parameters; and
transmit the brain stimulation protocol to the input/output arrangement.

2. The wearable device of claim 1, wherein the headwear arrangement further comprises a flexible cushioning member supporting the plurality of electrodes, wherein the flexible cushioning member maintains a physical contact between the plurality of electrodes and the skin of the user.

3. The wearable device of claim 1, wherein the processing unit is configured to analyse the electrical signals with a predetermined reference data set, by extracting the one or more signal features.

4. The wearable device of claim 1, wherein the at least one processing algorithm maps the one or more signal features to the one or more stimulation parameters to provide a balance between a sympathetic division and a parasympathetic division of a nervous system.

5. The wearable device of claim 3, wherein the data processing arrangement further comprises a memory module that stores the predetermined reference data set, wherein the processing unit is further configured to iteratively update the predetermined reference dataset in the memory module, when applying the at least one processing algorithm.

6. The wearable device of claim 1, wherein, when in operation, the input/output arrangement is configured to receive the electrical signals from the at least one of the plurality of electrodes concurrently with generating and applying the brain stimuli to the at least one of the plurality of electrodes.

7. The wearable device of claim 1, wherein, when in operation, the processing unit is configured to analyse the electrical signals with a predetermined reference data set concurrently with the application of the brain stimuli to the at least one of the plurality of electrodes, by configuring the input/output arrangement.

8. The wearable device of claim 1, wherein the processing unit is further configured to monitor one or more physiological markers corresponding to the brain stimuli, the one or more physiological markers comprising at least one of: a heart rate variability parameter, a beat-to-beat interval, a ratio of low frequency to high frequency heart rate variability.

9. The wearable device of claim 1, wherein the at least one processing algorithm comprises at least one of: at least one adaptive learning algorithm, at least one computational algorithm.

10. The wearable device of claim 9, wherein the at least one adaptive learning algorithm comprises at least one of: a K-nearest neighbour algorithm, a regression analysis, an ensemble tree based algorithms, maximum power point tracking, an artificial neural network, a deep convolutional neural network, a recurrent neural network, a reinforcement learning algorithm, a random forest algorithm, a recommender system, genetic algorithm, a Q-learning, a deep Q-learning algorithm, and a Bayesian optimisation algorithm.

11. The wearable device of claim 1, wherein the wearable device comprises a headset.

12. The wearable device of claim 1, wherein the plurality of electrodes are flexible, said plurality of electrodes being configured to adapt to a contour of the skin of the user, when the headwear arrangement is worn over the head of the user.

13. The wearable device of claim 2, wherein the flexible cushioning member comprises any one of: a silicon pad, a gel pad, a foam pad.

14. The wearable device of claim 2, wherein the flexible cushioning member comprises a conductive medium to provide electrical coupling between the plurality of electrodes and the skin of the user.

15. The wearable device of claim 2, wherein the flexible cushioning member comprises a porous electrolyte carrier saturated with saline, the porous electrolyte carrier being configured to maintain electrical contact between each of the plurality of electrodes and the skin of the user.

16. The wearable device of claim 1, further comprising a control unit communicably coupled with the data processing arrangement, wherein, when in operation, the control unit is configured to:
receive input from at least one user device; and
transmit the input to the processing unit of the data processing arrangement,
wherein the processing unit of the data processing arrangement is further configured to:
receive the input from the control unit;
update the brain stimulation protocol to generate an updated brain stimulation protocol, based on the received input; and
transmit the updated brain stimulation protocol to the input/output arrangement, and
wherein the control unit comprises a communication module that is configured to establish a communication link between the processing unit and a processor of the at least one user device.

17. The wearable device of claim 1, further comprising an external stimulation arrangement for providing at least one of: a visual stimulation, an audio stimulation, a virtual reality stimulation, concurrently with the delivery of the brain stimuli to the at least one of the plurality of electrodes.

18. The wearable device of claim 1, wherein the input/output arrangement comprises a safety arrangement configured to disable applying the brain stimuli to the plurality of electrodes and receiving the electrical signals from the plurality of electrodes, upon detection of at least one abnormal operating condition.

19. A method for monitoring a brain activity and providing non-invasive stimulation, the method being implemented by a wearable device, the method comprising:
positioning a headwear arrangement over a head of a user, the headwear arrangement comprising a plurality of electrodes configured to make electrical contact with skin of the user lying at least partially in a mastoid region of the user;
receiving, by the input/output arrangement that is operably coupled with the plurality of electrodes, electrical signals from at least one of the plurality of electrodes, wherein the electrical signals correspond to at least one of: brain activity of the user, heart activity of the user, vagus nerve activity of the user;
transmitting, by the input/output arrangement, the electrical signals to the data processing arrangement;
analysing, by the data processing arrangement, the electrical signals;
applying, by the data processing arrangement, at least one processing algorithm to map the one or more signal features to one or more stimulation parameters in a brain stimulation protocol;
generating, by the data processing arrangement, the brain stimulation protocol comprising the one or more stimulation parameters;
transmitting, by the data processing arrangement, the brain stimulation protocol to the input/output arrangement; and
generating and applying, by the input/output arrangement, brain stimuli to the at least one of the plurality of electrodes using the brain stimulation protocol, the brain stimuli being applied on the skin lying at least partially in the mastoid region.

20. The method of claim 19, wherein the data processing arrangement further comprises a memory module that stores a predetermined reference data set, the method further comprising iteratively updating the predetermined reference dataset in the memory module, when applying the at least one processing algorithm.
